(19) United States
(12) Patent Application Publication
(10) Pub. No.: US 2023/0302300 A1
(43) Pub. Date: Sep. 28, 2023

(54) MICROFABRICATED ULTRASOUND TRANSDUCER ARRAY FOR NEURAL STIMULATION

(71) Applicant: UNM Rainforest Innovations, Albuquerque, NM (US)

(72) Inventor: Nathan Morrow Jackson, Rio Rancho, NM (US)

(21) Appl. No.: 18/041,531

(22) PCT Filed: Sep. 13, 2021
(86) PCT No.: PCT/US2021/071436

§ 371 Date: Feb. 13, 2023

Related U.S. Application Data
(60) Provisional application No. 63/078,002, filed on Sep. 14, 2020.

(51) Int. Cl. A61B 5/00; B06B 1/06; A61B 5/293; B06B 1/02; A61N 7/00; B01L 3/00

(52) U.S. Cl. CPC A61N 7/00; A61B 5/293; A61B 5/4839; B01L 3/502715; B06B 1/0292; B06B 1/0629; B06B 1/0666; A61B 2562/028; A61B 2562/046; A61N 2007/0026; A61N 2007/0047; A61N 2007/0078; B01L 2300/0645; B01L 2300/0819; B01L 2400/0439; B06B 2201/76

(57) ABSTRACT

Apparatus, systems, and methods associated with microfabricated ultrasound transducer array for neural stimulation are applicable in a variety of applications. Microfabricated micro-scale ultrasound transducers can be integrated with microelectrode arrays for high spatial stimulation of neurons. Ultrasound stimulation of neurons can be combined with electrical recording to monitor neural activity. Such high spatial stimulation devices can be implemented for in-vitro and in-vivo applications. In-vivo applications can include high frequency ultrasound transducers incorporated into brain probes that are implanted at a specific area in the brain, where the high frequency ultrasound transducer can stimulate neurons at that target location.

### CLAIM OF PRIORITY

This application claims the priority benefit of U.S. Provisional Application Serial No. 63/078,002, filed 14 Sep. 2020, entitled “MICROFABRICATED ULTRASOUND TRANSDUCER ARRAY FOR NEURAL STIMULATION AND METHODS OF FABRICATION,” which application is incorporated herein by reference in its entirety.

### BACKGROUND

According to data from the World Health Organization nearly one billion people around the world suffer from some type of neurological disorder. Many of these could be relatively minor conditions such as chronic headaches. However, approximately fifty million people worldwide suffer from epilepsy and nearly ten million people have Parkinson’s disease, and this is increasing by approximately 60,000 cases per year. Most neurological disorders are treated through non-invasive methods such as pharmaceuticals. However, in cases where the patients’ symptoms are debilitating to the point where the patient can no longer function and drugs, such as L-dopa, are not working, an implanted device is used to help alleviate symptoms. The majority of these implanted devices are based on neurostimulation, which uses electrical stimulation to relieve symptoms.

The therapeutic effects of electrical stimulation on the peripheral and central nervous system have been an acceptable medical practice for several decades. Early forms of electrical stimulation for therapeutic effects can be traced back almost 2000 years to the Roman Empire, where electric fish were used to alleviate pain. Since then, electrical stimulation was widely used in early parts of the 20^{th} century as an electroconvulsive therapy in psychiatric patients. In the last couple of decades, an extensive amount of research has been performed on electrical stimulation, ranging from functional electrical stimulation (FES), spinal cord stimulation, pacemakers, retinal stimulation, cochlear implants, to deep brain stimulators (DBSs).

DBSs were implanted in the first patients in 1987. Since that time, DBS has grown significantly both in research and industry, and has demonstrated considerable success at treating several debilitating neurological disorders such as: Parkinson’s disease, Tourette syndrome, severe depression, chronic pain, and epilepsy. It has also led to other electrical stimulating devices such as the Vagus nerve stimulator (VNS). DBS has been researched extensively over the past two decades and this has led to improvements in treating specific disorders, understanding how DBS alleviates the symptoms, and efforts to determine if there are any long term neuroprotective effects. However, even with extensive amounts of research DBS still has some significant disadvantages including poor spatial resolution.

### BRIEF DESCRIPTION OF THE DRAWINGS

Embodiments of the invention are illustrated by way of example and not limitation in the figures of the accompanying drawings in which:

FIGS. 1A-1C are schematics of various stimulation approaches and the attenuation of the signals with respect to a stimulation area of a brain, in accordance with various embodiments.

FIGS. 2A-2D show example components to form a device for in-vitro applications, in accordance with various embodiments.

FIGS. 3A-3B are schematic representations of an example in-vivo probe implanted in a brain to stimulate a target region, in accordance with various embodiments.

FIGS. 4A-4B illustrate a theory of ultrasound stimulation from a hypothesized mechanism of action, in accordance with various embodiments.

FIG. 5 is a cross section schematic of in-vitro ultrasound stimulation device, in accordance with various embodiments.

FIG. 6 illustrates a system that can include an in-vitro ultrasound stimulation device for growing neurons and monitoring their electrical activity along with recording and stimulation capabilities, in accordance with various embodiments.

FIGS. 7A-7B illustrate examples of two different types of ultrasound transducers, in accordance with various embodiments.

FIG. 8 illustrates multiple piezoelectric micro-ultrasound transducer devices on a platform fabricated using aluminum nitride as a piezoelectric element, in accordance with various embodiments.

FIGS. 9A-9H illustrate an example fabrication process of a piezoelectric micro-ultrasound transducer device, in accordance with various embodiments.

FIG. 10 is a flow diagram of features of an example therapeutic method including neural stimulation, in accordance with various embodiments.

FIG. 11 is a flow diagram of an example method of forming an ultrasound stimulation device, in accordance with various embodiments.

FIG. 12 a block diagram illustrating components of an example system that can implement algorithms and perform methods structured to operate in conjunction with one or more ultrasound stimulation devices, in accordance with various embodiments.

### DETAILED DESCRIPTION

The following detailed description refers to the accompanying drawings that show, by way of illustration and not limitation, various example embodiments. These embodiments are described in sufficient detail to enable those skilled in the art to practice these and other embodiments. In order to avoid obscuring embodiments, some well-known system configurations and process steps are not disclosed in detail. Other embodiments may be utilized, and structural, logical, mechanical, and electrical changes may be made to these embodiments. The various embodiments are not necessarily mutually exclusive, as some embodiments can be combined with one or more other embodiments to form new embodiments. The following detailed description is, therefore, not to be taken in a limiting sense.

Therapeutic effects of electrical stimulation on the peripheral and central nervous system have been an acceptable medical practice for several decades and is widely used to treat disorders such as Parkinson’s disease, pain, depression, epilepsy, and various other medical issues. Electrical stimulation is currently the gold standard method for stimulating neurons. The high demand for electrical stimulation methods is partly due to the device simplicity, as electrical circuits to apply electrical stimulation are relatively less complex to manufacture as compared to other modes of stimulation such as light or conventional ultrasound. However, electrical stimulation in the brain can generate unwanted side effects due to its low spatial resolution. Electrical stimulation has low spatial resolution because the brain is conducting; therefore, electrical pulses transmitted in the brain due not attenuate, which causes unwanted neural stimulation in areas outside the desired location, which can lead to adverse side effects.

Electrical stimulation is the method conventionally used in DBS because it has been the most extensively researched stimulation method throughout history, and it is relatively easy to implement within a functional device. However, the use of electrical stimulation to treat neurological disorders, in particular Parkinson’s disease, can generate adverse side effects due to low spatial resolution. As noted above, electrical stimulation has low spatial resolution in the brain due to the high electrical conductivity of brain tissue. It has been reported that grey matter of the brain has a conductivity of approximately 0.2 S/m and white matter of the brain has a conductivity between 1 S/m and 0.1 S/m depending on orientation. Since the electrical conductivity in brain tissue is high, this results in poor spatial resolution for electrical stimulation because current propagates through the tissue.

In the case of Parkinson’s disease, the DBS electrodes are typically implanted in the subthalamic nucleus (STN), which is about 20-30 mm³ in size in humans. DBS electrode placement is difficult and is one of the major challenges associated with DBS. Even when the electrode is placed in the optimal location, adverse side effects can often occur. It has been reported that adverse side effects occur in approximately 4-17% of patients. These side effects have been reported by numerous research groups and have been tested using finite element modelling (FEM) methods. The adverse side effects are due to stimulation of neurons outside the target area (STN), and this is caused by electrical stimulations and their poor spatial resolution in brain tissue. Side effects can include involuntary movement, speech disturbances, ocular deviation, and muscle contraction. High density electrodes and smaller electrodes could be developed to help alleviate these problems; however, since the stimulation mechanism is still based on electrical methods, the stimulation will still propagate to tissue outside of the target area. In contrast to electrical stimulation that is due to current and does not attenuate quickly, ultrasound stimulation of neurons is caused from mechanical effects of pressure waves from ultrasound on neurons, which attenuates faster.

Recently, alternative methods of stimulating neurons have been validated including ultrasound and light. Ultrasound has been used extensively in medical applications for the past half century. Most people associate ultrasound with imaging because this is the most common application for medical ultrasound. It has been widely used across a range of applications from imaging of pregnant women, to imaging blood vessels via a tip on a catheter. Ultrasound also has therapeutic effects, usually related to heating or agitating the body, and typically uses much higher energy intensities. Therapeutic ultrasound applications include cleaning teeth, cancer treatment, physical therapy, kidney stone removal, cataract treatment, bone growth stimulation, and disrupting the blood brain barrier for drug delivery applications.

However, validation of ultrasound stimulation has been demonstrated on macro-scale (mm scale) ultrasound transducers that typically operate at low frequency (< 1 MHz). Typically, these devices are designed for external (non-invasive) stimulation, where focused arrays of transducers are used externally to drive and focus ultrasound waves to a particular region of the brain. These devices use low frequency transducers to penetrate the skin and skull to penetrate deep within the brain. Low frequency ultrasound does not attenuate quickly and has a high wavelength, which allows it to penetrate deep structures. Since low frequency does not attenuate, low frequency ultrasound also suffers from low spatial resolution. Focused arrays have been developed to try and resolve the poor spatial resolution, but often focused arrays use complex systems. High frequency transducers can produce much higher spatial resolution, but stimulus from high frequency transducers, while being applied externally, attenuate much quicker. Thus, the stimulus from these external high frequency transducers cannot penetrate to deep structures like in DBS.

In various embodiments, microfabricated micro-scale high frequency ultrasound transducers can be integrated with microelectrode arrays (MEAs) for high spatial stimulation of neurons. Ultrasound stimulation of neurons can be combined with electrical recording to monitor neural activity. Such high spatial stimulation devices can be implemented for in-vitro and in-vivo applications. High frequency ultrasound transducers can be incorporated into brain probes that are implanted at a specific area in the brain, where the high frequency ultrasound transducer can stimulate neurons at that target location such that the stimulation effectively attenuates without affecting other neurons outside of the target area. Arrays of transducers can be integrated into the probes to create enhanced spatial resolution in two dimensions by having a low wavelength, while the out of plane dimension spatial resolution is determined by the attenuation.

In various embodiments, micro-scale high frequency ultrasound transducers integrated onto a microelectrode array can be implemented for in-vivo or in-vitro applications. The microfabricated ultrasound transducers (MUTs) can be either capacitive micro-ultrasound transducers (CMUTs) or piezoelectric micro-ultrasound transducers (PMUTs). In operation of a PMUT or a CMUT, a membrane in transmission mode is driven to vibrate to transmit ultrasonic waves at the frequency of modulation and, in receiving mode, reflected ultrasonic waves incident on the membrane cause the membrane of the device to vibrate, which vibration can be detected.

For in-vivo applications, example embodiments can include micro-ultrasound transducers integrated into a silicon (Si) probe for DBS and combined with conducting electrodes for neural recordings, as well as microfluidic structures for drug delivery applications. Appropriate materials other than Si can be used for the probe.

Ultrasound devices integrated into an in-vitro MEA can be implemented to target stimulation of specific neurons or areas of brain slices for neuroscience research. In-vitro MEA are commonly used in the neuroscience research where neurons are extracted from brains and grown on the MEA substrate to monitor neural activity. Additionally, brain slices can also be used on a MEA substrate to monitor neural activity. Ultrasound stimulation using such devices, as taught herein, can also be applied to other applications, such as but not limited to cortical stimulation, peripheral stimulation, and cochlear stimulation. In addition to stimulation, ultrasound excitation can be used to prevent gliosis by breaking up scar tissue that forms around the electrodes due to inflammation process.

Ultrasound devices, as taught herein, can be fabricated using various techniques, which can include forming the ultrasound devices having a thin film membrane that displaces due to an applied electrical bias. The frequency of the vibration is dependent on the size and material stiffness of the membrane. The displacement due to an applied electrical bias can occur by using a piezoelectric film on the membrane or by using capacitive methods. Various piezoelectric materials can be used such as aluminum nitride (AlN), zinc oxide (ZnO), lead zirconate titanate (PZT), polyvinylidene fluoride (PVDF), lead magnesium niobate (PMN)-lead titanate (PT), or other similar compositions. The devices can be manufactured on various substrates such as, but not limited to, silicon or polymers.

Example approaches can be directed to two device types: in-vivo devices and in-vitro devices. For in-vivo devices, approaches, as taught herein, focus on using an implantable probe that can be implanted at the target brain region, with the tip of the implantable probe having an array of micro-ultrasound transducers (for example, either CMUT or PMUT). The ultrasound transducer can be fabricated using a substrate that is a commonly used material for implantable probes. For example, silicon is a commonly used material for implantable probes. The membrane for the ultrasound transducer can be developed by etching through the back-side of the substrate to create a membrane. A thin film piezoelectric film can be deposit for the membrane to provide actuation for the ultrasound transducer device. Recording electrodes, such as conductive polymers or metals, can be deposited and patterned to combine with an array of ultrasound transducers and electrodes. Microfluidic structures can be added for drug delivery using various techniques.

Micro-scale ultrasound transducers can have high resonant frequency due to the dimensions of these transducers. High frequency ultrasound attenuates quickly so it can provide high spatial resolution. High frequency ultrasound has a shorter wavelength than low frequency ultrasound, which allows high frequency ultrasound to provide a more precise target region. Spatial resolution of stimulation can be improved by using high frequency ultrasound stimulation (HFUS), because the wavelength of HFUS is low (i.e., typically 150 µm for 10 MHz compared to 1.5 mm for 1 MHz). Since high frequency attenuates more than low frequency ultrasound, impact of the stimulation effects around the target site can be reduced. Both of these characteristics can lead to a smaller volume of tissue activation (VTA), which can lead to less side effects.

For in-vitro devices, an array of microfabricated ultrasound transducers can be fabricated on a Si chip. This chip can be integrated onto a MEA. MEA devices can include an array of conducting electrodes that can monitor neural activity from a single neuron. However, these devices are typically made from Si or glass which is not acoustically transparent, so it does not allow ultrasound waves to transmit through the substrate. Therefore, a polymer with conducting electrodes can be used. Examples of such polymers include, but are not limited to, polyimide and parylene. Spacers in between the ultrasound transducers and the MEA can be used to bond the two devices together. The MEA can include a fluidic channel that allows the space between the ultrasound and the MEA to be filled with liquid medium that is used to promote neural growth and to keep the cells alive. A separate fluidic chamber can be included as well. This allows ultrasound waves to propagate through the liquid and an underlying MEA substrate to stimulate cells on the MEA substrate. Such a device can offer increased spatial resolution due to the low wavelength, which affects the x and y spatial resolution. The z direction resolution is dependent on the attenuation of the waves in the medium. For high frequency application the attenuation is high, thus cells that are further away will be subjected to lower intensity ultrasound waves, which will not cause stimulation. This technique can be combined to form high spatial resolution stimulation of single neurons.

Stimulation techniques can focus on DBS; however, with the stimulation techniques being sufficiently reliable, these techniques can be used in other applications. In various embodiments, an example approach can include miniaturizing an ultrasound stimulator by using MEMS fabrication techniques and incorporating this ultrasound stimulator in an all-in-one neural interface device with one or more probes having stimulator and recording electrodes. A PMUT can be used as the stimulating device, because of its unique properties and its fabrication, which is typically easier to fabricate than other ultrasound transducers. However, other ultrasound transducers, such as but not limited to CMUTs, can be used. High frequency ultrasound can improve spatial resolution because the pressure waves caused from ultrasound attenuate in a short distance in brain tissue compared to low frequency ultrasound. Miniaturized ultrasound stimulators integrated in brain probes can be directed to improve the quality of life of patients suffering from debilitating neurological diseases such as Parkinson’s disease with reduced side effects compared to current DBS devices.

In various embodiments, high frequency piezoelectric micro-ultrasound transducers can be fabricated that are compatible with complementary metal-oxide-semiconductor (CMOS) technology. Such high frequency CMOS compatible piezoelectric micro-ultrasound transducers can be used to stimulate neurons to improve the spatial resolution of neurostimulation devices, in particular DBS.

Previously, researchers have shown that ultrasound can be used to stimulate neurons. Unlike electrical stimulation, the mechanism of action is believed to be due to mechanical stimulation from the pressure waves. Ultrasound is a high frequency pressure wave that travels through a medium and is classified as a sound wave operating above 20 kHz. Although undetectable to the human ear, ultrasound has been applied across a wide spectrum of applications, perhaps the most commonly known is foetal imaging but applications such as non-invasive neurosurgery are also emerging.

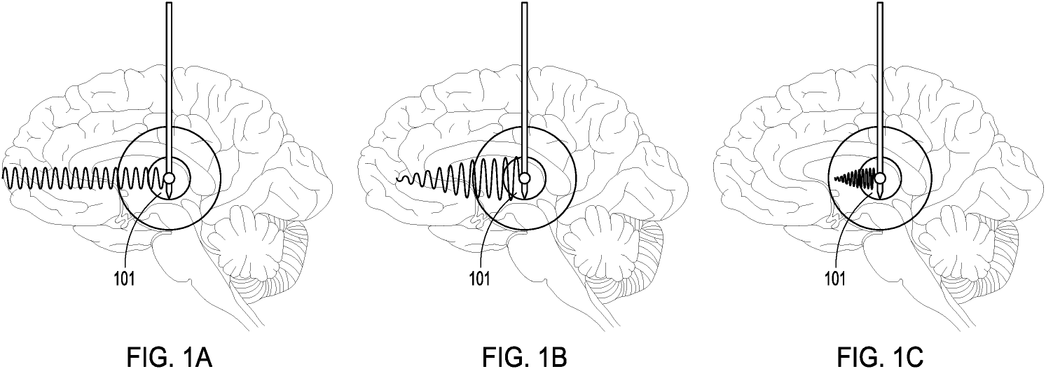

Low frequency ultrasound (< 1 MHz) has the advantage that it can penetrate into deep structures because the attenuation coefficient in Newtonian fluids is proportional to the square of frequency. Tissue has the properties of both a porous solid and a fluid. High frequency ultrasound (1-200 MHz) on the other hand has a shorter wavelength and has higher attenuation. Attenuation occurs as the ultrasound energy is absorbed by the media in which it is travelling. The amount of attenuation is based on an attenuation coefficient (α), which is directly linked to the frequency. For example, a 1 MHz ultrasound wave has an a of 0.85 dB/cm, where a 10 MHz ultrasound signal has a value of approximately 10 dB/cm in brain tissue. This means that a 1 MHz wave’s power will reduce by 10 dB (or about 10 times the intensity (W/cm²)) in about 11.7 cm, where a 10 MHz wave will reduce or attenuate the same amount in about 1 cm. Higher frequencies will attenuate even more rapidly. Visualization of improving spatial resolution is shown schematically in FIGS. 1A-1C.

FIGS. 1A-1C are schematics of various stimulation approaches and the attenuation of the signals with respect to stimulation area 101. Stimulation area 101 is overestimated for illustration purposes. FIG. 1A illustrates a typical electrical stimulation to stimulation area 101. FIG. 1B illustrates low frequency ultrasound stimulation to stimulation area 101, where the signal attenuates after a significant length. FIG. 1C illustrates high frequency stimulation, where the waves are attenuated within the target region thus improving spatial resolution. FIGS. 1A-1C are not drawn to scale and basic sine waves are shown to represent the range of stimulation. These figures are meant to represent the attenuation of the waves and how this can be used to improve spatial resolution. By improving spatial resolution, the adverse side effects should be reduced.

High frequency ultrasound can also improve the lateral resolution because the focus of an ultrasound device is improved as the wavelength gets smaller, which is directly linked to the frequency. The wavelength is directly linked to the frequency through the formula λ=c/f, where λ is the wavelength, c is the speed of sound, and f is the frequency. For instance, a 200 MHz ultrasound transducer produces a wavelength of about 7 µm in brain tissue, and a 15 MHz transducer would produce a wavelength of about 100 µm, showing that the higher the frequency, the higher is the lateral precision. This precision provides an impetus to use high frequency ultrasound to stimulate neurons and improve spatial resolution because it can lead to development of a system to precisely target neuron clusters for stimulation. Ultrasonic beam-steering for the devices developed can have performance characteristics vastly superior to currently pursued stimulation methods. Ultrasound can be used to create lesions using very high intensities of (> 600 W/cm²), so safety is always a concern when using high frequency ultrasound. However, previous intensities used to stimulate neurons range from 50-300 mW/cm², which is in the range of imaging ultrasound. The stimulation intensity can also be defined by the mechanical index (MI), which is an ultrasound metric that takes into account the negative pressure and the frequency. Currently, the Food and Drug Administration (FDA) approved limit is 1.9.

As noted above, high frequency ultrasound can improve spatial resolution both axially and laterally, thus improving the overall volume tissue activation. Axially, it is improved due to attenuation through the media, and laterally, it is improved due to the wavelength of the ultrasound. High frequency ultrasound is an advantage because higher frequency waves attenuate quicker and have a lower wavelength. An array of PMUTs can be designed to stimulate larger lateral areas. The micro-machined ultrasound transducers can be made using MEMS fabrication technology. The PMUT can be fabricated using CMOS compatible processing, which allows inclusion of advanced circuitry such as amplifiers and wireless telemetry systems. The size of the PMUTs, in this example, can range between 10-100 µm in diameter and can be designed to operate as a single PMUT design or an array of transducers.

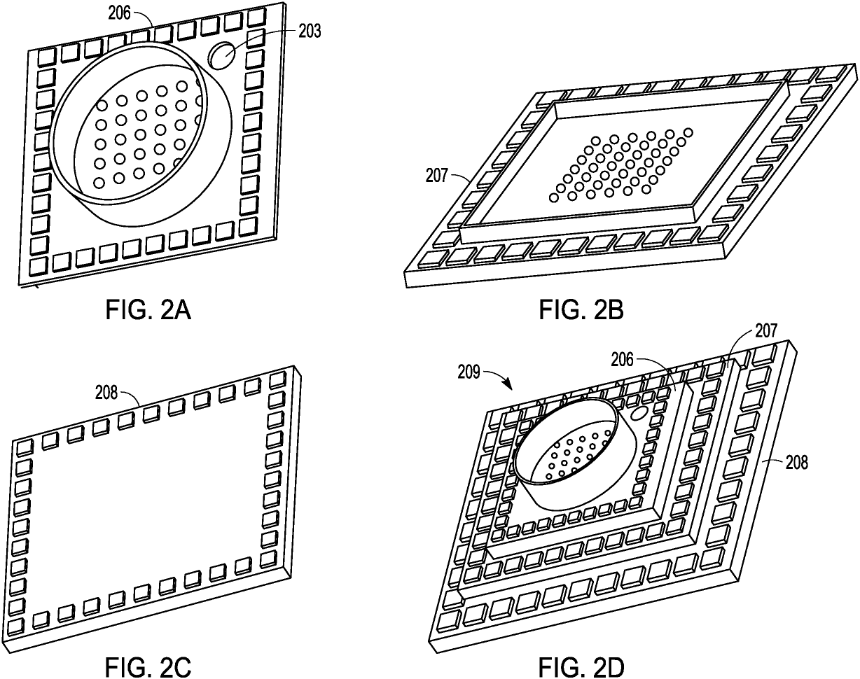

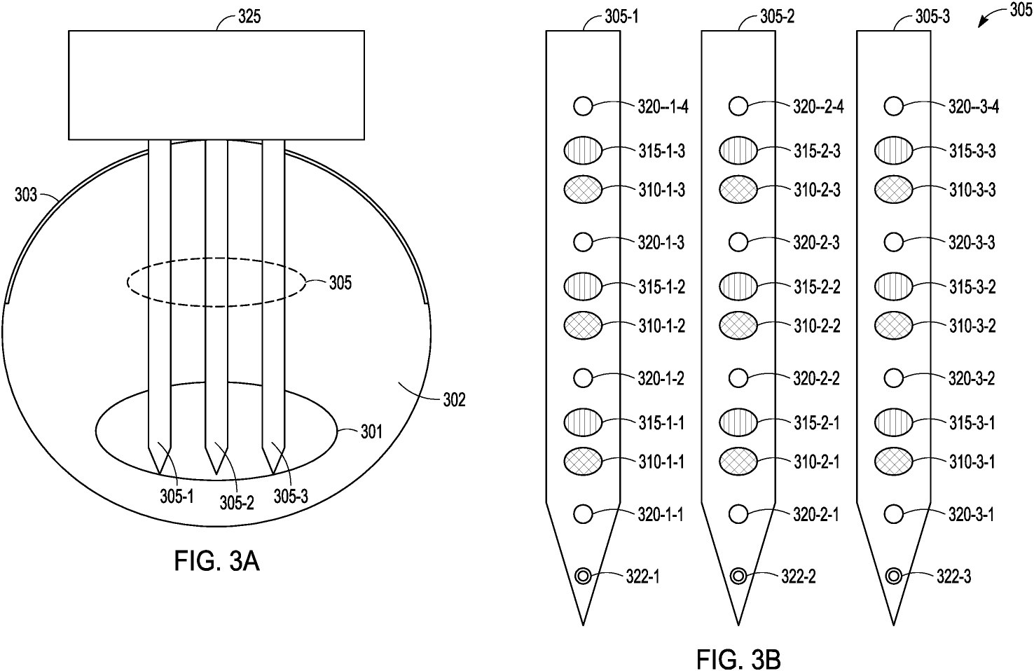

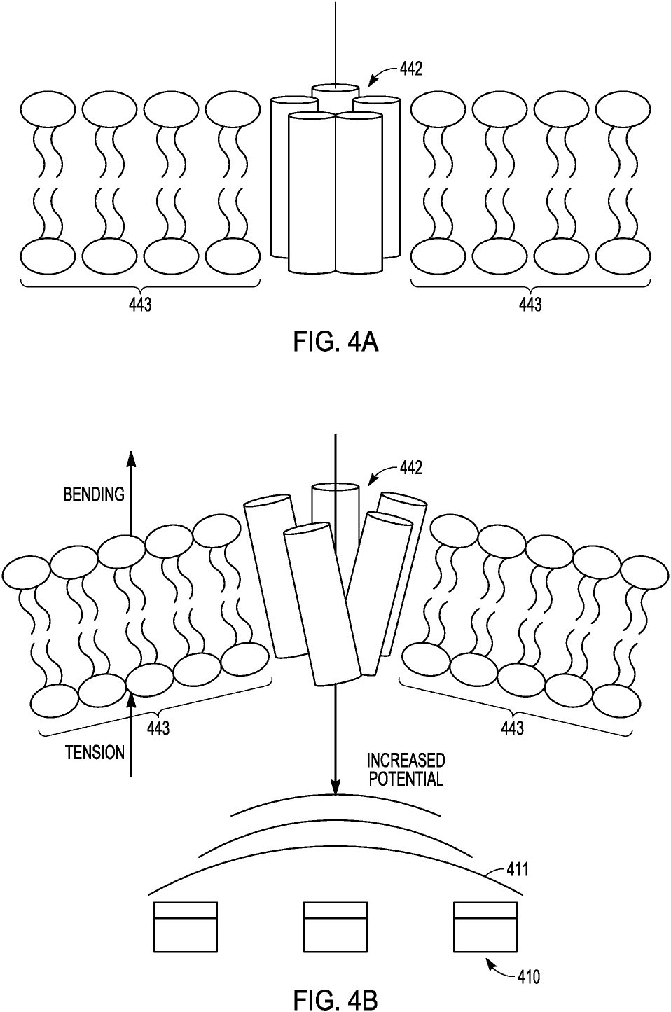

FIGS. 2A-2D show an embodiment of example components to form a device for in-vitro applications. FIG. 2A is a representation of an in-vitro MEA component 206 containing an acoustically transparent MEA for recording, where the MEA includes a fluid inlet 203. FIG. 2B is a representation of an ultrasonic array 207, which can be formed as a PMUT array or a CMUT array. FIG. 3C is a presentation of a printed circuit board (PCB) 208 on which ultrasonic array 207 and in-vitro MEA component 206 can be disposed. FIG. 4D is a presentation of an integration 209 of in-vitro MEA component 206, ultrasonic array 207, and PCB 208 to form a complete device for in-vitro applications.

FIGS. 3A-3B are schematic representations of an embodiment for an example in-vivo probe 305 implanted in a brain 302 to stimulate a target region 301. In-vivo probe 305 can be implemented as one or more probes. In the example of FIGS. 3A-3B, in-vivo probe 305 includes probes 305-1, 305-2, and 305 -3, though in-vivo probe 305 can be structured with more or less than three probes. As represented in FIG. 3A, in-vivo probe 305 can be inserted through skull 303 of a patient or other subject into brain 302. In-vivo probe 305 can be coupled to control circuitry 325 that is located exterior to skull 303, where control circuitry 325 can control application of stimulus to target region 301 and collection of data from in-vivo probe 305. The control circuitry 325 can be configured on a platform to be located on skull 325.

Control circuitry 325 can include wireless capability for communicating to a remote machine. The remote machine, not shown in FIG. 3A, can perform analysis from neural recordings of data captured by in-vivo probe 305. The neural recordings can allow monitoring of neural activity of the subject. Such a remote machine can also provide parameters for control circuitry 325 to operate in-vivo probe 305, which can allow control circuitry 325 to make adjustments to in-vivo probe 305. The adjustments can include adjusting or re-activating activation of one or more probes 305-1, 305-2, and 305 -3 of in-vivo probe 305 in response to the monitoring of the neural activity.

Control circuitry 325 can include wired communication capability for communicating to a remote machine. Control circuitry 325 can include one or more memory devices to store data collected from in-vivo probe 305 for later communication to the remote machine at a time in which control circuitry 325 is connected for wired communication. With control circuitry 325 connected for wired communication, control circuitry 325 can transmit data received from in-vivo probe 305. Control circuitry 325 can include analysis capability to report to a remote machine. With such analysis capability, control circuitry 325 can monitor neural activity of the subject and make adjustments to components of in-vivo probe 305.

FIG. 3B illustrates arrangements of components on probes 305-1, 305-2, and 305-3 of in-vivo probe 305 of FIG. 3A. Probe 305-1 can include, on a platform that is compatible with implantation in a living subject, ultrasound transducers 310-1-1, 310-1-2, and 310-1-3 and electrical stimulating electrodes 315-1-1, 315-1-2, and 315-1-3 along with recording electrodes 320-1-1, 320-1-2, 320-1-3, and 320-1-4. Though three ultrasound transducers are shown, probe 305-1 can have more or less than three ultrasonic transducers. Though three electrical stimulating electrodes are shown, probe 305-1 can have more or less than three electrical stimulating electrodes. Though four recording electrodes are shown, probe 305-1 can have more or less than four recording electrodes.

Probe 305-2 can include, on a platform that is compatible with implantation in a living subject, ultrasound transducers 310-2-1, 310-2-2, and 310-2-3 and electrical stimulating electrodes 315-2-1, 315-2-2, and 315-2-3 along with recording electrodes 320-2-1, 320-2-2, 320-2-3, and 320-2-4. Though three ultrasound transducers are shown, probe 305-2 can have more or less than three ultrasonic transducers. Though three electrical stimulating electrodes are shown, probe 305-2 can have more or less than three electrical stimulating electrodes. Though four recording electrodes are shown, probe 305-2 can have more or less than four recording electrodes.

Probe 305-3 can include, on a platform that is compatible with implantation in a living subject, ultrasound transducers 310-3-1, 310-3-2, and 310-3-3 and electrical stimulating electrodes 315-3-1, 315-3-2, and 315-3-3 along with recording electrodes 320-3-1, 320-3-2, 320-3-3, and 320-3-4. Though three ultrasound transducers are shown, probe 305-3 can have more or less than three ultrasonic transducers. Though three electrical stimulating electrodes are shown, probe 305-3 can have more or less than three electrical stimulating electrodes. Though four recording electrodes are shown, probe 305-3 can have more or less than four recording electrodes.

In some applications, probes 305-1, 305-2, and 305-3 can include outlets 322-1, 322-2, and 322-3, respectively, from microfluidic channels in the respective probes. Outlets 322-1, 322-2, and 322-3 can be used to deliver a drug from the microfluidic channel in the respective probes, as managed by control circuitry 325 to which the probes are coupled. Outlets 322-1, 322-2, and 322-3 can be arranged at the tip of the probes 305-1, 305-2, and 305-3, respectively. Each of probes 305-1, 305-2, and 305-3 can include multiple microfluidic channels, with each microfluidic channel having an outlet or with the multiple microfluidic channels sharing an outlet.

Initial analysis can be performed for a device containing one or more PMUTs, recording components, and electrical stimulating electrodes with respect to a comparison on spatial resolution and safety determination. The in-vivo microelectrode probes can be approximately 100-200 µm wide, 5-9 mm in length to reach deep structures in brain 302. The PMUT’s can be on the order of 10-100 µm diameter for both the in-vitro and in-vivo devices. Other sizes can be implemented.

The stimulation parameters for high frequency ultrasound device can be determined; however initial values can be based on previous results. The frequency of stimulation of the PMUT should not be confused with the frequency for electrical stimulation. Electrical stimulation frequency refers to how often a stimulus pulse is given. Frequency in ultrasound represents the frequency of the sound waves, which can be given as a continuous (DC stimulation in electrical) or pulsed stimulation. High frequency stimulation refers to the frequency of the ultrasound, the actual pulsed stimulation frequency can be determined but is believed to be similar to electrical stimulation (~130 Hz).

Ultrasound has an extensive history in the medical field. However, most of the research and development of systems have been focused around imaging for diagnostic applications. Ultrasound transducers convert electrical signals into oscillating pressure waves and vice versa. High intensity ultrasound can also be used to damage tissue (ablation at 600 W/cm²) and gene transfection through sonoporation (opening of pores of cell membrane for short duration). Therefore, operating directed towards optimizing the stimulation parameters can be critical to prevent unwanted damage to cells (typical values range from 50-300 mW/cm²).

In the past decade, there have been several cases of using ultrasound to stimulate neurons as a proof-of-concept, as it has the potential advantage of being a non-invasive method of stimulating deep tissue with increased spatial resolution. However, current technology does not allow researchers to understand the mechanism of action. Fabrication and integration of a MEMS-based ultrasound stimulation device and MEA is challenging, but essential to better understand how the stimulation operates. In order for ultrasound devices to advance from a proof-of-concept to a viable replacement of electrical stimulation, an understanding of the mechanism of action is important, as it will allow researchers to optimize the device for their specific applications.

Previous research on ultrasound stimulation used macro-scale (5-10 mm diameter) transducers to stimulate neurons in-vivo and in-vitro. In the case of in-vitro stimulation, the ultrasound transducer was typically placed above the MEA in the liquid media. The problem with this method is that it can cause contamination issues for long-term monitoring of neurons, and the macro-scale transducers stimulated the entire network of neurons. Stimulating the entire network of neurons prevents the researcher from understanding the mechanism of action; however, it can be useful for determining stimulation parameters. Recent studies, conducted by the inventor, solved the problem of contamination by placing the transducer on the bottom of the MEA. This technique used a polymer MEA substrate. However, this method still stimulates the entire network of neurons since it used macro-scale transducers.

In various embodiments, an array of MUT devices with dimensions of about 10 µm to about 50 µm can be able to focus the sound waves by using beam forming, which will be able to target a single cell. An integrated MEMS device with high spatial resolution can be used to better understand the mechanism of action of ultrasound stimulation.

The mechanism of action of ultrasound stimulation is unknown, but possible mechanisms include cavitation, heating, and mechanical stimulation. Heating and cavitation are unlikely to be the primary mechanism because they require high intensities of ultrasound. The hypothesis is that ultrasound stimulation is due to mechanical effects. Over the last two decades molecular neuroscientists have discovered specific ion channels that open when mechanically stimulated by mechanoreceptors. Mechanoreceptors relay extracellular stimulus from outside a cell to signal transduction within a cell through mechanically gated ion channels. The idea is that these ion channels act like piezoelectric elements. It has been demonstrated that most biological tissue and cell membranes have piezoelectric properties or more accurately ferroelectric properties, as they respond to heat as well as mechanical deformation due to the non-centrosymmetric structure of biological molecules. A theory of ultrasound stimulation from a hypothesized mechanism of action is illustrated in FIGS. 4A-4B.

FIG. 4A illustrates ion channels 443 about cell membrane 442 at rest. FIG. 4B illustrates ultrasound bending membrane 443 opening the ion channels 443 creating an action potential. A pressure wave 411 created from a MUT 410 creates a bending motion of the cell membrane 442 and ion channels 443, which causes cell membrane 442 to open up and increase its conductance, creating an action potential. The concept is that the cell membrane 442 and ion channels 443 function in the direct piezoelectric effect mode. The cell membrane 442 can also operate in converse piezoelectric effect mode by applying an electric field across the membrane (electrical stimulation method) which creates a deformation of the cell membrane 442.

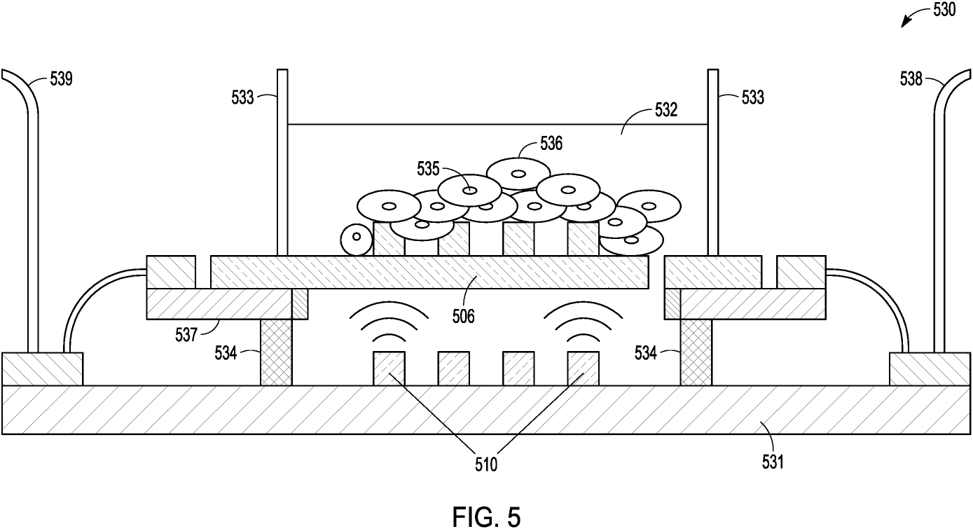

FIG. 5 is a cross section schematic of in-vitro ultrasound stimulation device 530. In-vitro ultrasound stimulation device 530 can include a glass well 533 to contain a culture solution 532 for neurons 535 being investigated. Neurons 535 can be grown in culture solution 532 or can be included in brain slices 536 in culture solution 532. Neurons 535 and brain slices 536 can be disposed on a MEA substrate 506 having low acoustic attenuation. MEA substrate 506 and glass well 533 can be coupled to a platform 531 via a rigid substrate 537 and spacer 534. A micro-ultrasound transducer 510 can be dispose on platform 531 below MEA substrate 506. Platform 531 can provide a mechanism to couple MEA substrate 506 to an excitation input 538 and a mechanism to couple MEA substrate 506 to an MEA readout 539.

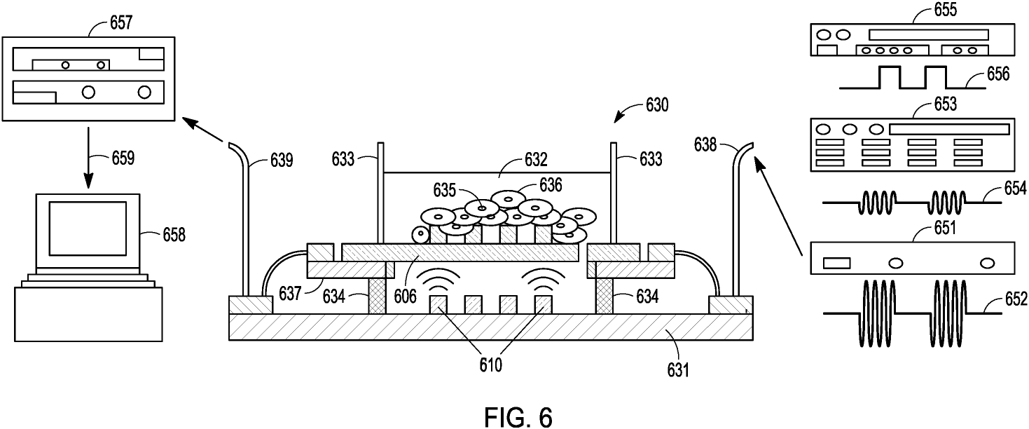

FIG. 6 illustrates a system that can include an in-vitro ultrasound stimulation device 630 for growing neurons and monitoring their electrical activity. In-vitro ultrasound stimulation device 630 can be implemented similar to or identical to in-vitro ultrasound stimulation device 530 of FIG. 5. In-vitro ultrasound stimulation device 630 can include a glass well 633 to hold a culture solution 632 for neurons 635 being investigated. Neurons 635 can be grown in culture solution 632 or can be included in brain slices 636 in culture solution 632. Neurons 635 and brain slices 636 can be disposed on a MEA substrate 606 having low acoustic attenuation. MEA substrate 606 and glass well 633 can be coupled to a platform 631 via a rigid substrate 637 and spacer 634. A micro-ultrasound transducer 610 can be disposed on a platform 631 below MEA substrate 606. Platform 631 can provide a mechanism to couple MEA substrate 606 to an excitation input 638 and a mechanism to couple MEA substrate 606 to an MEA readout 639.

Excitation input 638 can receive a stimulation signal to stimulate neurons 635. The stimulation signal can be created using a pulse generator 655 to provide a pulse signal 656 that can be used by a radio frequency (RF) signal generator 653 to generate a RF signal 654 . The generated RF signal 654 can be coupled to a RF amplifier 651 to generate an amplified RF signal 652 for excitation input 638.

MEA readout 639 can be coupled to an electrophysiology recording system 657. The coupling from MEA readout 639 to electrophysiology recording system 657 can be wired or wireless. MEA readout 639 can communicate, via a communication mechanism 659, with a computing system 658 to analyze the responses from stimulating neurons 635. Computing system 658 can be remote from in-vitro ultrasound stimulation device 630. Computing system 658 can be remote from electrophysiology recording system 657. The communication mechanism 659 can be a wired transmission mechanism, a wireless transmission mechanism, or a combination of a wired transmission mechanism and a wireless transmission mechanism.

The MEA device of in-vitro ultrasound stimulation device 630 can be made from a thin film polymer substrate in order to allow the ultrasound waves to transmit through low acoustic attenuation MEA substrate 606 and onto the neurons 635. MEA devices can be realized in a number of different types. One type of MEA devices can be structured as a planar device. A second type of MEA devices can include nanopillars for molecular monitoring, where the nanopillars can be used to mechanically deform the cell membrane causing an action potential. Manipulators can be used in such devices. MUT devices, such as implemented for micro-ultrasound transducer 610, can be bonded below the MEA device and can include an array of MUTs with various frequencies in order to determine effects of ultrasound frequencies. The various frequencies can range from about 0.1 MHz to about 100 MHz. Low frequencies are used for non-invasive clinical applications; however, high frequencies can be used to improve spatial resolution to investigate fundamental neuroscience. Higher frequencies are used for single cell stimulation. The individual MUT devices can be fabricated with dimensions similar to the size of a neuron, which is approximately between 10 µm and 50 µm diameters). When placed in an array, the MUT devices can focus on a single cell.

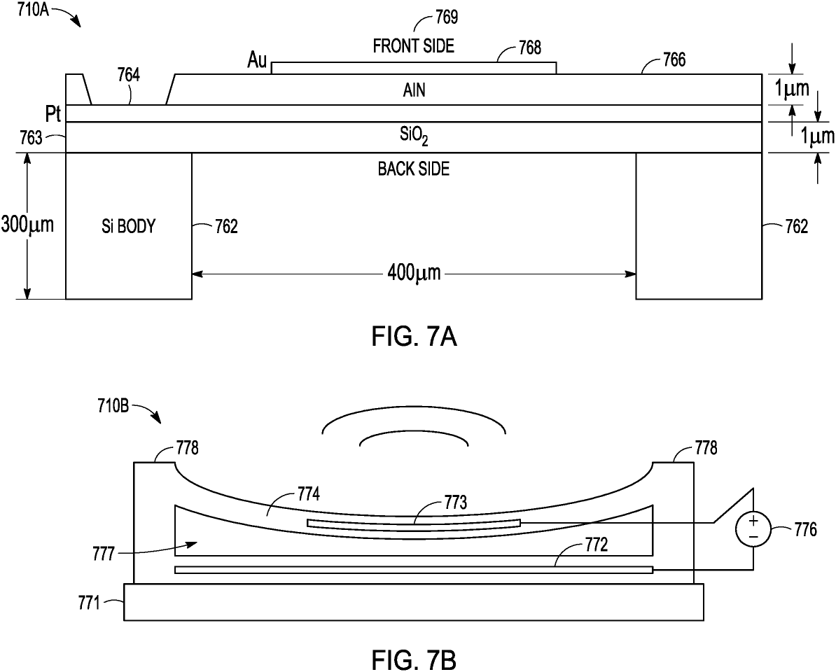

FIGS. 7A-7B illustrate embodiments of two different types of ultrasound transducers. FIG. 7A shows a cross-sectional view of an embodiment of an example PMUT 710-A that is based on piezoelectric elements. FIG. 7B shows a cross-sectional view of an embodiment of an example CMUT 710-B. Researchers that used macro-scale devices to stimulate neurons typically used PMUTs, as they are commercially available. Both types of transducers can be developed for different applications, such as chemical or biological sensing applications. CMUTs operating at frequencies as high as 50 MHz have been developed for such chemical and biological sensing applications.

PMUT 710-A can include a Si body 762 having a portion removed to provide an opening for a back side 761 of PMUT 710-A. In this non-limiting example, the opening can have a width of approximately 400 µm in Si body 762, with Si body 762 having a height of approximately 300 µm. A SiO₂ layer 763 can be structured on Si body 762 directly above and covering the opening in Si body 762. SiO₂ layer 763 can have a thickness of approximately one µm. A Pt layer 764 can be disposed on and contacting SiO₂ layer 763. Pt layer 764 can have a width extending over the entire opening in Si body 762. Pt layer 764 can have a thickness of less than one µm. A Ti layer can be used instead of a Pt layer. An A1N layer 766 can be disposed on and contacting Pt layer 764. A1N layer 766 can have a width extending over the entire opening in Si body 762. In this example, A1N layer 766 is shorter than Pt layer 764, providing an access region to Pt layer 764. A1N layer 766 can have a thickness of approximately one µm. An Au layer 768 can be disposed on and contacting A1N layer 766. Au layer 768 can have a width less than the opening in Si body 762. Au layer 768 can be centered with respect to the opening in Si body 762. Au layer 768 provides a front side 769 of PMUT 710-A. Other materials and dimensions can be used for an integrated PMUT device.

CMUT 710-B can include a doped Si substrate 771 on which is disposed a dielectric frame 778. A dielectric membrane 774 extends across a vacuum region 777 in dielectric frame 778. Dielectric membrane 774 can be a silicon nitride member. Dielectric membrane 774 can include a metal electrode 773 disposed above vacuum region 777. On a side of vacuum region 777 opposite metal electrode 773 is a metal electrode 772 in dielectric frame 778 above doped Si substrate 771. Metal electrode 773 and metal electrode 772 can be coupled to a voltage supply 776. Metal electrode 773 and metal electrode 772 can be implemented with aluminum electrodes. Other materials and dimensions can be used for an integrated CMUT device.

MUTs, such as PMUTs and CMUTs, can be formed and used in molecular monitoring, cellular monitoring, and finite element modelling (FEM) that can be used in a computing system, such as computing system 658, to analyze the responses from stimulating neurons. An integrated MEMS ultrasound device with MEA can be capable of delivering a better understanding of the mechanism of action of ultrasound with respect to neuron stimulation.

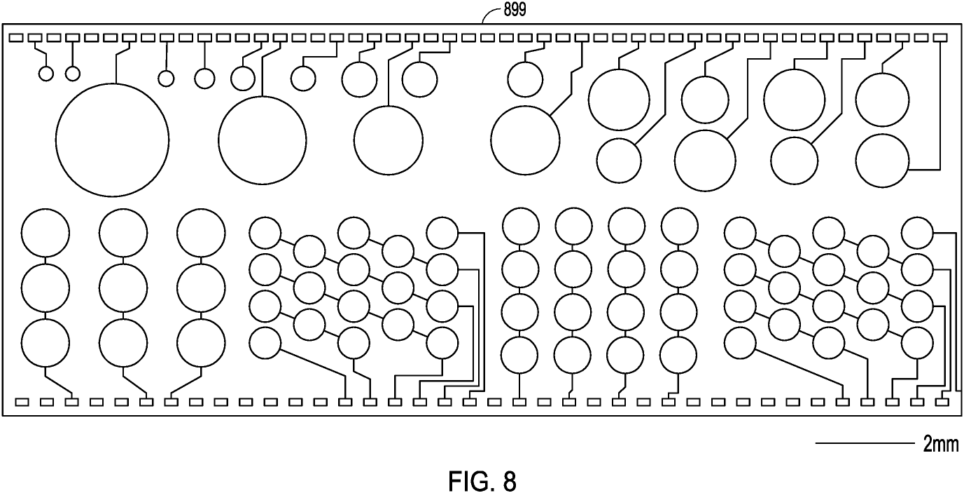

In various embodiments, MUT devices can be implemented structured as an array of MUTs based on PMUT or CMUT designs. Polyimide MEAs can be used since MEA devices made from silicon or Pyrex are typically not acoustically transparent. The use of nanopillars can be implemented onto MEA devices for molecular testing. Both MEA and MUT devices can be implemented using materials to enhance the properties of the devices, such as piezoelectric properties for the PMUT. Conducting polymers that are more acoustically transparent than typical metal electrodes can be used. Integrating MEA and MUT devices can provide a new approach to neuron stimulation and investigation. For example, FIG. 8 shows multiple PMUT devices using AlN as a piezoelectric element fabricated on a platform 899.

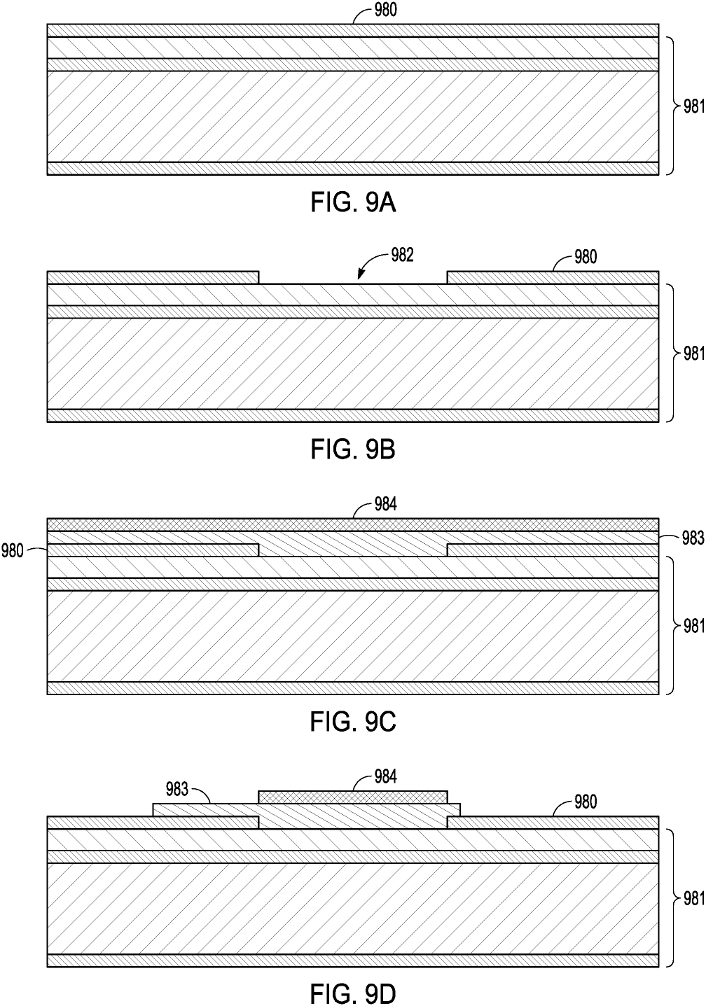

FIGS. 9A-9H are an illustration of an embodiment of an example fabrication process of a PMUT device. FIG. 9A shows a silicon-on-insulator (SOI) 891 after an oxide 980 has been formed on SOI 891. Formation of oxide 980 can be performed using an appropriate deposition process such as but not limited to chemical vapor deposition (CVD), atomic layer deposition (ALD), or other deposition process. FIG. 9B shows the structure of FIG. 9A after oxide 980 has been etched to form a region for a diaphragm. FIG. 9C shows the structure of FIG. 9B after a Ti layer 983 and an AlN layer 984 have been formed. Formation of Ti layer 983 and AlN layer 984 can be performed using an appropriate deposition process. FIG. 9D shows the structure of FIG. 9C after Ti layer 983 and AlN layer 984 are patterned.

FIG. 9E shows the structure of FIG. 9D after forming dielectric regions 885 on top of the structure of FIG. 9D. FIG. 9F shows the structure of FIG. 9E after forming conductive contact region 986 on Ti layer 983 and conductive contact region 987 on AlN layer 984. FIG. 9G shows the structure of FIG. 9F after etching top oxide 840 and a Si region of SOI 891 to shape diaphragm regions. FIG. 9H shows the structure of FIG. 9G after etching the backside of SOI 891 using deep reactive-ion etching (DRIE) to define cavity 989. Other materials can be used in fabrication of PMUT devices similar to the formed PMUT device of FIGS. 9A-9H.

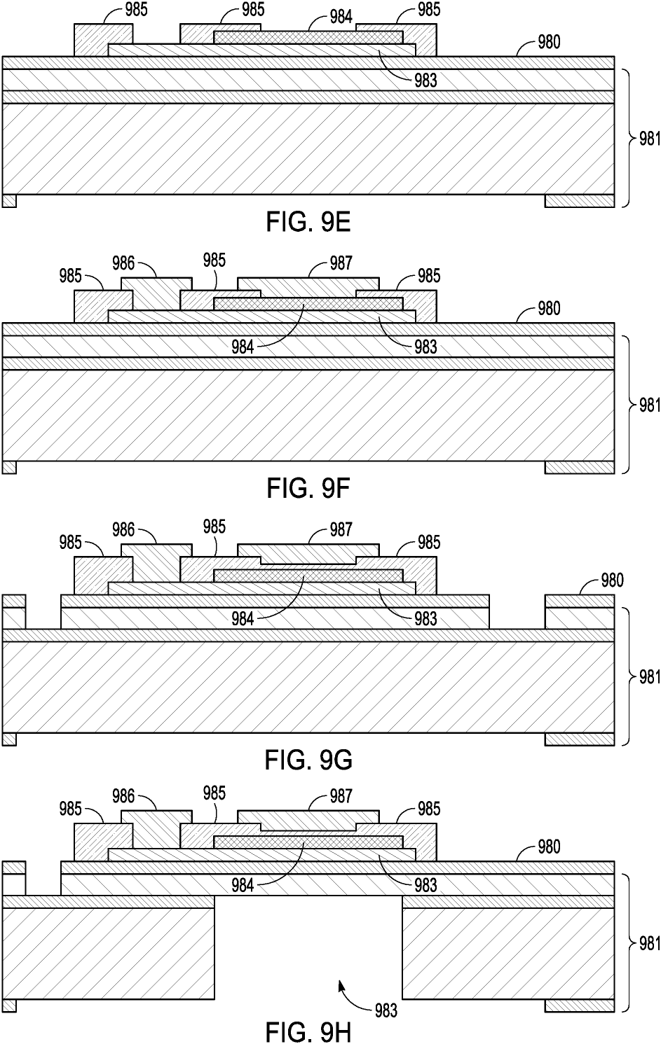

FIG. 10 is a flow diagram of features of an embodiment of an example therapeutic method 1000 including neural stimulation. At 1010, a probe is implanted at a target brain region of a subject, with the probe having an array of micro-ultrasound transducers integrated with a microelectrode array on the probe. The implanted probe can be coupled to control circuitry to control operation of the probe. The implanted probe can be structured as multiple individual probes, either with individual control circuitry or control circuitry to control operation of the multiple individual probes. The implanted probe may be structured similar to probe 305 of FIGS. 3A-3B. At 1020, one or more micro-ultrasound transducers of the array of micro-ultrasound transducers are activated.

Variations of method 1000 or methods similar to method 1000 can include a number of different embodiments that may be combined depending on the application of such methods and/or the architecture of devices or systems in which such methods are implemented. Such methods can include making neural recordings of signals received by the array of micro-ultrasound transducers in response to the activating of the one or more micro-ultrasound transducers. Neural activity of the subject can be monitored from the neural recordings. In response to the monitoring of the neural activity, the one or more micro-ultrasound transducers can be adjusted or re-activated. Such adjustments can include changing amplitude of the activation signal, frequency of the activation signal to the one or more micro-ultrasound transducers, selection of specific probes of the one or more micro-ultrasound transducers, or other parameters associated with conducting neural stimulation using the one or more micro-ultrasound transducers. In the situation in which none of the one or more micro-ultrasound transducers are currently being driven, an activation signal, based on the monitoring of the neural activity, can be applied to re-activate the one or more micro-ultrasound transducers. The adjustment or re-activation of the one or more micro-ultrasound transducers can be managed by control circuitry for the implanted probe. The control circuitry can be exterior to the subject, such as control circuitry 325 of FIG. 3A. The control circuitry can operate in conjunction with communication with a remote computing system such as computing system 658 of FIG. 6.

Variations of method 1000 or methods similar to method 1000 can include delivering a drug using one or more microfluidic channels in the probe. The delivery can be managed by control circuitry for the implanted probe. The control circuitry for drug delivery can be exterior to the subject, such as control circuitry 325 of FIG. 3. Such control circuitry can operate in conjunction with communication with a remote computing system such as computing system 658 of FIG. 6. Control circuitry for drug delivery and control circuitry for adjustment and activation of the implanted probe can be implemented as a single integrated structure. With respect to method 1000 and variations thereof, the array of micro-ultrasound transducers can be operable to generate sound waves having a frequency in a range from approximately 1 MHz to approximately 200 MHz.

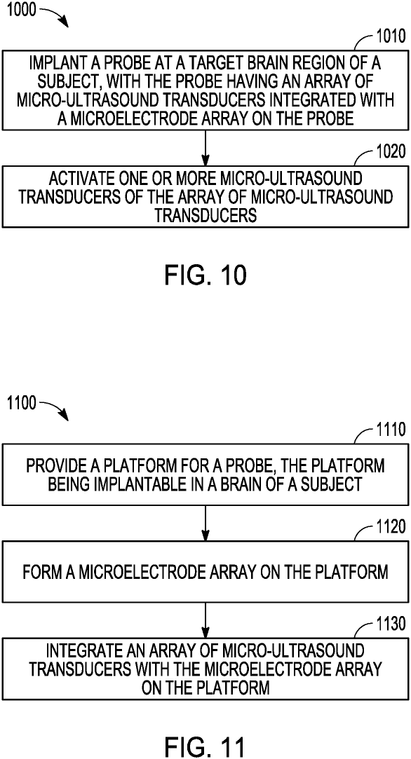

FIG. 11 is a flow diagram of an embodiment of an example method of forming an ultrasound stimulation device. At 1110, a platform is provided for a probe, with the platform being implantable in a brain of a subject. The platform is selected to be formed with material compatible for implantation in a living subject. For example, the platform can be a silicon-based substrate. At 1120, a microelectrode array is formed on the platform. At 1130, an array of micro-ultrasound transducers is integrated with the microelectrode array on the platform.

Variations of method 1100 or methods similar to method 1100 can include a number of different embodiments that may be combined depending on the application of such methods and/or the architecture of devices or systems in which such methods are implemented. Such methods can include integrating micro-ultrasound transducers having membranes that displace in response to an applied electrical bias for the micro-ultrasound transducers of the array of micro-ultrasound transducers. Such variations can include each membrane being arranged as component of a capacitor of the micro-ultrasound transducer in which the membrane is disposed. Variations can include forming a piezoelectric film on each membrane. The piezoelectric film on each membrane can include one or more of AlN, ZnO, PZT, PVDF, or PMN-PT on the membrane.

With respect to method 1100 and variations thereof, material of the membranes of integrated micro-ultrasound transducers can have a size and material stiffness to operate the micro-ultrasound transducers to generate sound waves having a frequency in a range from approximately 1 MHz to approximately 200 MHz. The integrated micro-ultrasound transducers and the microelectrode array can be formed with components, sizes, and materials for various ultrasound stimulation devices as taught herein.

In various embodiments, an ultrasound stimulation device can comprise one or more probes implantable in a brain of a subject. Each probe of the one or more probes can include an array of micro-ultrasound transducers and a microelectrode array integrated with the array of micro-ultrasound transducers on the probe.

Variations of such an ultrasound stimulation device can include a number of different embodiments that may be combined depending on the application of such ultrasound stimulation devices and/or the architecture in which such ultrasound stimulation devices are implemented. Such ultrasound stimulation device can be structured with the array of micro-ultrasound transducers including capacitive micro-ultrasound transducers or piezoelectric micro-ultrasound transducers. With the micro-ultrasound transducers of the array of micro-ultrasound transducers being piezoelectric micro-ultrasound transducers, piezoelectric material of the piezoelectric micro-ultrasound transducers can include one or more of AlN, ZnO, PZT, PVDF, or PMN-PT.

Variations of such an ultrasound stimulation device can include the microelectrode array having electrical stimulating electrodes and recording electrodes. The recording electrodes can include conductive polymers. Variations can include the array of micro-ultrasound transducers having a silicon substrate. The one or more probes can be silicon-based probes. Variations can also include at least one probe of the one or more probes having one or more microfluidic channels arranged to deliver a drug to a target location in the brain of the subject to which the ultrasound stimulation device is applied.

Variations of such an ultrasound stimulation device can include the ultrasound stimulation device having control circuitry to couple to the one or more probes. The control circuitry can be disposed on a platform attachable to an exterior of the subject. The ultrasound stimulation device and variations thereof can be structured such that the array of micro-ultrasound transducers and the control circuitry are operable to generate sound waves having a frequency in a range from approximately 1 MHz to approximately 200 MHz. The ultrasound stimulation device and variations thereof can be structured with components, sizes, materials, and additional instrumentalities as taught herein.

In various embodiments, an ultrasound stimulation device can comprise an array of micro-ultrasound transducers on a chip, a microelectrode array having an array of conducting electrodes, with the microelectrode array integrated onto the chip, and a fluidic channel to contain a neuron sample or a brain slice on a substrate of the microelectrode array. The fluidic channel can be arranged to allow space between the micro-ultrasound transducers and the microelectrode array to be filled with a liquid.

Variations of such an ultrasound stimulation device can include a number of different embodiments that may be combined depending on the application of such ultrasound stimulation devices and/or the architecture in which such ultrasound stimulation device can include spacers between the array of micro-ultrasound transducers and the microelectrode array. The spacers can be structured to bond the array of micro-ultrasound transducers and the microelectrode array together. Variations can include the chip being a silicon chip. Material of the fluidic channel can include a polymer. Variations of such an ultrasound stimulation device can include the array of micro-ultrasound transducers having capacitive micro-ultrasound transducers or piezoelectric micro-ultrasound transducers.

The ultrasound stimulation device and variations thereof can be structured such that the micro-ultrasound transducers are structured within the ultrasound stimulation device to generate sound waves having a frequency in a range from approximately 1 MHz to approximately 200 MHz. The ultrasound stimulation device and variations thereof can be structured with components, sizes, materials, and additional instrumentalities as taught herein.

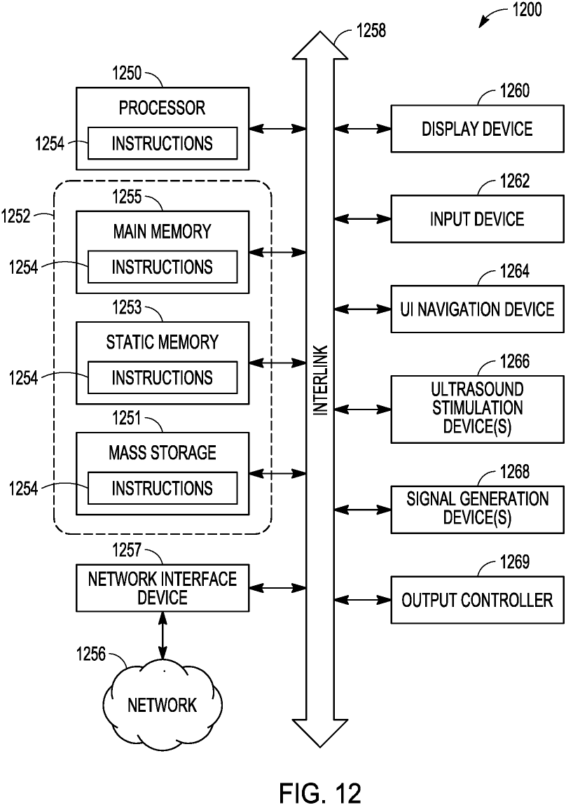

FIG. 12 is a block diagram illustrating components of an embodiment of an example system 1200 that can implement algorithms and perform methods structured to operate in conjunction with one or more ultrasound stimulation devices 1266 and associated methods as taught herein. System 1200 can include one or more processors 1250 that can be structured to execute stored instructions to perform functions to control, manage, or use output of one or more ultrasound stimulation devices 1266. Ultrasound stimulation devices 1266 can include microfabricated ultrasound transducer arrays integrated with microelectrode arrays for neural stimulation.

System 1200 may operate as a standalone system or may be connected, for example networked, to other systems. In a networked deployment, system 1200 can operate in the capacity of a server machine, a client machine, or both in server-client network environments. In an example, system 1200 can act as a peer machine in peer-to-peer (P2P) (or other distributed) network environment. Further, while only a single system is illustrated, the term “system” shall also be taken to include any collection of systems that individually or jointly execute a set (or multiple sets) of instructions to perform any one or more of the methodologies discussed herein.

System 1200 can be a machine (e.g., a computer-based machine) and can include a hardware processor 1250 (e.g., a CPU, a GPU, a hardware processor core, or any combination thereof), a main memory 1255, and a static memory 1253, some or all of which can communicate with each other via components of an interlink (e.g., bus) 1258. The interlink 1258 can include a number of different communication mechanisms such as different wired communication mechanisms and different wireless communication mechanisms. System 1200 can further include a display device 1260, an alphanumeric input device 1262 (e.g., a keyboard), and a user interface (UI) navigation device 1264 (e.g., a mouse). In an example, display device 1260, input device 1262, and UI navigation device 1264 can be a touch screen display. System 1200 can additionally include a mass storage device (e.g., drive unit) 1251, one or more signal generation devices 1268. System 1200 can include other sensors, such as a global positioning system (GPS) sensor, compass, accelerometer, or other communication-enabled sensors. System 1200 can include an output controller 1269, such as a serial (e.g., USB, parallel, or other wired or wireless (e.g., infrared (IR), near field communication (NFC), etc.) connection to communicate or control one or more peripheral devices (e.g., a printer, card reader, etc.).

System 1200 can include a machine-readable medium 1252 on which is stored one or more sets of data structures or instructions 1254 (e.g., software) embodying or utilized by system 1200 to perform any one or more of the techniques or functions for which system 1200 is designed. Instructions 1254 can also reside, completely or at least partially, within main memory 1255, within static memory 1253, or within hardware processor 1250 during execution thereof by system 1200. In an example, one or any combination of hardware processor 1250, main memory 1255, static memory 1253, or mass storage device 1251 can constitute machine-readable medium 1252.

Instructions 1254 (e.g., software, programs, an operating system (OS), etc.) or other data can be stored on the mass storage device 1251 and can be accessed by main memory 1255 for use by processor 1250. Main memory 1255 (e.g., DRAM) is typically fast, but volatile, and thus a different type of storage than mass storage device 1251 (e.g., an SSD), which is suitable for long-term storage, including while in an “off” condition. Instructions 1254 or data in use by a user or system 1200 are typically loaded in main memory 1255 for use by processor 1250. When main memory 1255 is full, virtual space from mass storage device 1251 can be allocated to supplement main memory 1255; however, because mass storage device 1251 is typically slower than main memory 1255, and write speeds are typically at least twice as slow as read speeds, use of virtual memory can greatly reduce user experience due to storage device latency (in contrast to main memory 1255, e.g., DRAM). Further, use of mass storage device 1251 for virtual memory can greatly reduce the usable lifespan of mass storage device 1251.

Instructions 1254, data, results of data analysis, or parameters for neural stimulation and monitoring of responses from neural stimulation can be transmitted or received over a network 1256 using a transmission medium via a network interface device 1257 utilizing any one of a number of transfer protocols (e.g., frame relay, Internet protocol (IP), transmission control protocol (TCP), user datagram protocol (UDP), hypertext transfer protocol (HTTP), power transfer protocols etc.). Example network 1256 can include one or more communication networks having a local area network (LAN), a wide area network (WAN), a packet data network (e.g., the Internet), mobile telephone networks (e.g., cellular networks), Plain Old Telephone (POTS) networks, and wireless data networks, among others. In an example, the network interface device 1257 can include one or more physical jacks (e.g., Ethernet, coaxial, or phone jacks) or one or more antennas to connect to the communications network 1256. In an example, the network interface device 1257 can include a plurality of antennas to wirelessly communicate using at least one of single-input multiple-output (SIMO), multiple-input multiple-output (MIMO), or multiple-input single-output (MISO) techniques. The term “transmission medium” shall be taken to include any tangible non-transitory medium that is capable of carrying instructions or data to and for execution by the machine 1200 and includes instrumentalities to propagate digital or analog communications signals to facilitate communication of such instructions, which instructions can be implemented by software.

The following are example embodiments of an ultrasound stimulation device and associated methods, in accordance with the teachings herein.

An example ultrasound stimulation device 1 can comprise: one or more probes implantable in a brain of a subject, each probe of the one or more probes including: an array of micro-ultrasound transducers; and a microelectrode array integrated with the array of micro-ultrasound transducers on the probe.

An example ultrasound stimulation device 2 can include features of example ultrasound stimulation device 1 and can include the array of micro-ultrasound transducers including capacitive micro-ultrasound transducers or piezoelectric micro-ultrasound transducers.

An example ultrasound stimulation device 3 can include features of any of the preceding example ultrasound stimulation devices and can include the micro-ultrasound transducers of the array of micro-ultrasound transducers being piezoelectric micro-ultrasound transducers.

An example ultrasound stimulation device 4 can include features of any of the preceding example ultrasound stimulation devices and can include piezoelectric material of the piezoelectric micro-ultrasound transducers including one or more of AlN, ZnO, PZT, PVDF, or PMN-PT.

An example ultrasound stimulation device 5 can include features of any of the preceding example ultrasound stimulation devices and can include the microelectrode array including electrical stimulating electrodes and recording electrodes.

An example ultrasound stimulation device 6 can include features of any of the preceding example ultrasound stimulation devices and can include the recording electrodes including conductive polymers.

An example ultrasound stimulation device 7 can include features of any of the preceding example ultrasound stimulation devices and can include the array of micro-ultrasound transducers including a silicon substrate.

An example ultrasound stimulation device 8 can include features of any of the preceding example ultrasound stimulation devices and can include the one or more probes being silicon-based probes.

An example ultrasound stimulation device 9 can include features of any of the preceding example ultrasound stimulation devices and can include at least one probe of the one or more probes includes one or more microfluidic channels arranged to deliver a drug.

An example ultrasound stimulation device 10 can include features of any of the preceding example ultrasound stimulation devices and can include the ultrasound stimulation device including control circuitry to couple to the one or more probes.

An example ultrasound stimulation device 11 can include features of any of the preceding example ultrasound stimulation devices and can include the array of micro-ultrasound transducers and the control circuitry being operable to generate sound waves having a frequency in a range from approximately 1 MHz to approximately 200 MHz.

An example ultrasound stimulation device 12 can include features of any of the preceding example ultrasound stimulation devices and can include the control circuitry being disposed on a platform attachable to an exterior of the subject.

An example ultrasound stimulation device 13 can comprise: an array of micro-ultrasound transducers on a chip; a microelectrode array having an array of conducting electrodes, with the microelectrode array integrated onto the chip; and a fluidic channel to contain a neuron sample or a brain slice on a substrate of the microelectrode array, with the fluidic channel arranged to allow space between the micro-ultrasound transducers and the microelectrode array to be filled with a liquid.

An example ultrasound stimulation device 14 can include features of example ultrasound stimulation device 13 and can include the ultrasound stimulation device including spacers between the array of micro-ultrasound transducers and the microelectrode array, the spacers structured to bond the array of micro-ultrasound transducers and the microelectrode array together.

An example ultrasound stimulation device 15 can include features of any of the preceding example ultrasound stimulation devices 13-14 and can include the chip being a silicon chip.

An example ultrasound stimulation device 16 can include features of any of the preceding example ultrasound stimulation devices 13-15 and can include material of the fluidic channel including a polymer.

An example ultrasound stimulation device 17 can include features of any of the preceding example ultrasound stimulation devices 13-16 and can include the array of micro-ultrasound transducers including capacitive micro-ultrasound transducers or piezoelectric micro-ultrasound transducers.

An example ultrasound stimulation device 18 can include features of any of the preceding example ultrasound stimulation devices 13-17 and can include the micro-ultrasound transducers being structured within the ultrasound stimulation device to generate sound waves having a frequency in a range from approximately 1 MHz to approximately 200 MHz.

An example therapeutic method 1 can comprise activating one or more micro-ultrasound transducers of an array of micro-ultrasound transducers on a probe implanted at a target brain region of a subject, with the array of micro-ultrasound transducers integrated with a microelectrode array on the probe.

An example therapeutic method 2 can include features of example therapeutic method 1 and can include making neural recordings of signals received by the array of micro-ultrasound transducers in response to the activating of the one or more micro-ultrasound transducers.

An example therapeutic method 3 can include features of any of the preceding example therapeutic methods and can include monitoring neural activity of the subject from the neural recordings.

An example therapeutic method 4 can include features of any of the preceding example therapeutic methods and can include the therapeutic method including adjusting or re-activating the one or more micro-ultrasound transducers in response to the monitoring of the neural activity.

An example therapeutic method 5 can include features of any of the preceding example therapeutic methods and can include delivering a drug using one or more microfluidic channels in the probe.

An example therapeutic method 6 can include features of any of the preceding example therapeutic methods and can include the array of micro-ultrasound transducers generating sound waves having a frequency in a range from approximately 1 MHz to approximately 200 MHz.

An example method 1 of forming an ultrasound stimulation device can comprise: providing a platform for a probe, the platform being implantable in a brain of a subject; forming a microelectrode array on the platform; and integrating an array of micro-ultrasound transducers with the microelectrode array on the platform.

An example method 2 of forming an ultrasound stimulation device can include features of example method 1 of forming an ultrasound stimulation device and can include integrating the array of micro-ultrasound transducers to include integrating micro-ultrasound transducers having membranes that displace in response to an applied electrical bias.

An example method 3 of forming an ultrasound stimulation device can include features of any of the preceding example methods of forming an ultrasound stimulation device and can include arranging each membrane as component of a capacitor of the micro-ultrasound transducer in which the membrane is disposed.

An example method 4 of forming an ultrasound stimulation device can include features of any of the preceding example methods of forming an ultrasound stimulation device and can include forming a piezoelectric film on each membrane.

An example method 5 of forming an ultrasound stimulation device can include features of any of the preceding example methods of forming an ultrasound stimulation device and can include forming the piezoelectric film on each membrane to include forming one or more of AlN, ZnO, PZT, PVDF, or PMN-PT on the membrane.

An example method 6 of forming an ultrasound stimulation device can include features of any of the preceding example methods of forming an ultrasound stimulation device and can include forming material of the membranes having a size and material stiffness to operate the micro-ultrasound transducers to generate sound waves having a frequency in a range from approximately 1 MHz to approximately 200 MHz

Applications of such devices and methods as taught herein can have potentially significant impact. There are over 10,000 people per year around the world who are implanted with DBS devices, and more than 40% of them suffer from major side effects. Microfabricated ultrasound transducer arrays or variations thereof can be used to reduce side effects associated with neural stimulation. Forms of these device can be utilized in in-vivo animal research and in in-vitro neuro research.

Although specific embodiments have been illustrated and described herein, it will be appreciated by those of ordinary skill in the art that any arrangement that is calculated to achieve the same purpose may be substituted for the specific embodiments shown. Upon studying the disclosure, it will be apparent to those skilled in the art that various modifications and variations can be made in the devices and methods of various embodiments. Various embodiments can use permutations and/or combinations of embodiments described herein. Other embodiments will be apparent to those skilled in the art from consideration of the specification and practice of the embodiments disclosed herein. It is to be understood that the above description is intended to be illustrative, and not restrictive, and that the phraseology or terminology employed herein is for the purpose of description.

What is claimed is:

1. An ultrasound stimulation device comprising:
one or more probes implantable in a brain of a subject, each probe of the one or more probes including:
an array of micro-ultrasound transducers; and
a microelectrode array integrated with the array of micro-ultrasound transducers on the probe.

2. The ultrasound stimulation device of claim 1, wherein the array of micro-ultrasound transducers includes capacitive micro-ultrasound transducers or piezoelectric micro-ultrasound transducers.

3. The ultrasound stimulation device of claim 2, wherein the micro-ultrasound transducers of the array of micro-ultrasound transducers are piezoelectric micro-ultrasound transducers.

4. The ultrasound stimulation device of claim 3, wherein piezoelectric material of the piezoelectric micro-ultrasound transducers includes one or more of aluminum nitride, zinc oxide, lead zirconate titanate, polyvinylidene fluoride, or lead magnesium niobate-lead titanate.

5. The ultrasound stimulation device of claim 1, wherein the microelectrode array includes electrical stimulating electrodes and recording electrodes.

6. The ultrasound stimulation device of claim 5, wherein the recording electrodes include conductive polymers.

7. The ultrasound stimulation device of claim 1, wherein the array of micro-ultrasound transducers includes a silicon substrate.

8. The ultrasound stimulation device of claim 1, wherein the one or more probes are silicon-based probes.

9. The ultrasound stimulation device of claim 1, wherein at least one probe of the one or more probes includes one or more microfluidic channels arranged to deliver a drug.

10. The ultrasound stimulation device of claim 1, wherein the ultrasound stimulation device includes control circuitry to couple to the one or more probes.

11. The ultrasound stimulation device of claim 10, wherein the array of micro-ultrasound transducers and the control circuitry are operable to generate sound waves having a frequency in a range from approximately 1 MHz to approximately 200 MHz.

12. The ultrasound stimulation device of claim 10, wherein the control circuitry is disposed on a platform attachable to an exterior of the subject.

13. A therapeutic method comprising:
activating one or more micro-ultrasound transducers of an array of micro-ultrasound transducers on a probe implanted at a target brain region of a subject, with the array of micro-ultrasound transducers integrated with a microelectrode array on the probe.

14. The therapeutic method of claim 13, wherein the therapeutic method includes making neural recordings of signals received by the array of micro-ultrasound transducers in response to the activating of the one or more micro-ultrasound transducers.

15. The therapeutic method of claim 14, wherein the therapeutic method includes monitoring neural activity of the subject from the neural recordings.

16. The therapeutic method of claim 15, wherein the therapeutic method includes adjusting or re-activating the one or more micro-ultrasound transducers in response to the monitoring of the neural activity.

17. The therapeutic method of claim 13, wherein the method includes delivering a drug using one or more microfluidic channels in the probe.

18. The therapeutic method of claim 13, wherein the method includes the array of micro-ultrasound transducers generating sound waves having a frequency in a range from approximately 1 MHz to approximately 200 MHz.

19. A method of forming an ultrasound stimulation device, the method comprising:
providing a platform for a probe, the platform being implantable in a brain of a subject;
forming a microelectrode array on the platform; and
integrating an array of micro-ultrasound transducers with the microelectrode array on the platform.

20. The method of claim 19, wherein integrating the array of micro-ultrasound transducers includes integrating micro-ultrasound transducers having membranes that displace in response to an applied electrical bias.

21. The method of claim 20, wherein each membrane is arranged as component of a capacitor of the micro-ultrasound transducer in which the membrane is disposed.

22. The method of claim 20, wherein the method includes forming a piezoelectric film on each membrane.

23. The method of claim 22, wherein forming the piezoelectric film on each membrane includes forming one or more of aluminum nitride, zinc oxide, lead zirconate titanate, polyvinylidene fluoride, or lead magnesium niobate-lead titanate on the membrane.

24. The method of claim 20, wherein the method includes forming material of the membranes having a size and material stiffness to operate the micro-ultrasound transducers to generate sound waves having a frequency in a range from approximately 1 MHz to approximately 200 MHz.

25. An ultrasound stimulation device comprising:
an array of micro-ultrasound transducers on a chip;
a microelectrode array having an array of conducting electrodes, with the microelectrode array integrated onto the chip; and
a fluidic channel to contain a neuron sample or a brain slice on a substrate of the microelectrode array, with the fluidic channel arranged to allow space between the micro-ultrasound transducers and the microelectrode array to be filled with a liquid.

26. The ultrasound stimulation device of claim 25, wherein the ultrasound stimulation device includes spacers between the array of micro-ultrasound transducers and the microelectrode array, the spacers structured to bond the array of micro-ultrasound transducers and the microelectrode array together.

27. The ultrasound stimulation device of claim 25, wherein the chip is a silicon chip.

28. The ultrasound stimulation device of claim 25, wherein material of the fluidic channel includes a polymer.

29. The ultrasound stimulation device of claim 25, wherein the array of micro-ultrasound transducers includes capacitive micro-ultrasound transducers or piezoelectric micro-ultrasound transducers.

30. The ultrasound stimulation device of claim 25, wherein the micro-ultrasound transducers are structured within the ultrasound stimulation device to generate sound waves having a frequency in a range from approximately 1 MHz to approximately 200 MHz.
